(19) United States
(12) Patent Application Publication
(10) Pub. No.: US 2015/0080878 A1
(43) Pub. Date: Mar. 19, 2015

(54) DEVICE AND METHOD FOR TISSUE REMOVAL

(71) Applicant: Board of Regents, the University of Texas System, Austin, TX (US)

(72) Inventors: Yusheng Feng, San Antonio, TX (US); Robert Svatek, San Antonio, TX (US); Mohammad Rahman, San Antonio, TX (US); John Jordan, San Antonio, TX (US); Luis Davila, San Antonio, TX (US)

(73) Assignee: Board of Regents, the University of Texas System, Austin, TX (US)

(21) Appl. No.: 14/391,891

(22) PCT Filed: Apr. 12, 2013
(86) PCT No.: PCT/US13/36467

§ 371 Date: Oct. 10, 2014

Related U.S. Application Data
(60) Provisional application No. 61/624,223, filed on Apr. 13, 2012.

(51) Int. Cl. A61B 18/08; A61B 17/3209

(52) U.S. Cl. CPC A61B 18/08; A61B 17/3209
USPC 606/37; 606/40

(57) ABSTRACT

Exemplary embodiments are directed to devices and methods for tissue removal. Exemplary embodiments can be configured for mechanical dissection as well as suction to grasp, resect and collect all or part of a target tissue. Exemplary embodiments may also comprise elements for cauterization of tissue and coagulation of blood vessels.

### CROSS-REFERENCE TO RELATED APPLICATION

This application claims priority to U.S. Provisional Patent Application Ser. No. 61/624,223 filed Apr. 13, 2012, the entire contents of which are incorporated herein by reference.

### STATEMENT REGARDING FEDERALLY FUNDED RESEARCH

This invention was made with government support under 5K25CA116291 awarded by the National Institutes of Health and HRD#0932339 awarded by National Science Foundation. The government has certain rights in the invention.

### BACKGROUND INFORMATION

Cancer spreading or metastasis is a severe condition that a surgeon would strive to control in order to save patients' lives. The cancer cells often travel through vascular or lymphatic systems to remote sites, which can induce secondary tumors. Excision of lymphatic tissue is an important component of many surgical operations. Termed “lymph node dissection”, this procedure provides prognostic value for patients and in some cases may prevent subsequent cancer metastasis. The conventional lymph node dissection involves surgical removal of lymphatic nodes and vessels near the primary tumors and in the regional lymphatic tissue that provides lymphatic drainage for the primary tumor.

The human body transports fluids and cells in the lymphatic and vascular systems to maintain normal and healthy functions. Cancer cells can infiltrate the lymphatic system and spread from an original tumor to a remote site. Once disseminated, eradication of cancer is usually not possible. Although chemotherapy may help slow the growth of cancer in metastatic disease, relapse and progression is common and death is inevitable in most solid tumors. In many solid tumors, the tumor is removed along with the surrounding lymphatic channels. This is done to determine if the cancer has penetrated the lymphatic system and in some cases it can eradicate the cancer within the lymphatic system. Termed lymph node dissection, these procedures are performed for many types of cancer including breast, prostate, stomach, uterine, cervical, urinary bladder, testicular, and others.

The commonly used technique for removing the lymphatic tissue is time consuming It requires intricate cutting and cauterizing using various surgical tools to grasp, resect, or collect the tissue. The tissue must be precisely cut to avoid damage to the surrounding arteries, veins, and nerve bundles. During the procedures the patient is anesthetized for prolonged period of times, which may put the patient at a higher risk for the adverse events and complications. Thus, there is an important need for more efficient techniques and devices for removing tissues, such as lymphatic tissue.

### SUMMARY

Exemplary embodiments of the present disclosure include a tissue removal device comprising: an elongated housing comprising a proximal end and a distal end; an electric motor; a fixed cauterizing element proximal to the distal end of the housing; and a rotating cauterizing element proximal to the distal end of the housing, where the electric motor is coupled to the rotating cauterizing element. In specific embodiments, the elongated housing may be generally cylindrical or close to cylindrical, and in certain embodiments the electric motor may be coupled to the rotating cauterizing element via a drive extension within the elongated housing.

Particular embodiments may comprise a drive member coupled to the electric motor and the drive extension. In certain embodiments, the fixed cauterizing element may comprise a fixed cutting element that is electrically conductive; the rotating cauterizing element may comprise a rotating cutting element that is electrically conductive; and the fixed cutting element may be in contact with the rotating cutting element. In specific embodiments, the tissue removal device may be configured so that an electric power source can be electrically coupled to the fixed cauterizing element. In particular embodiments, the fixed cutting element can be electrically coupled to the electric power source via a wire extending along a primary length of the elongated housing.

In certain embodiments, the tissue removal device may be configured so that radio frequency electric power is provided to the fixed cutting element. Particular embodiments may comprise a first control switch configured to supply electrical power to the fixed cutting element at a first power level sufficient to cauterize tissue. Specific embodiments can further comprise a second control switch configured to supply electrical power to the fixed cutting element at a second power level sufficient to coagulate blood vessels. In certain embodiments the wire may be located in a channel in the elongated housing.

In particular embodiments, the rotating cauterizing element may comprise a rotating cutting element holder that is not electrically conductive and the fixed cauterizing element may comprise a fixed cutting element holder that is not electrically conductive. In specific embodiments, the fixed cutting element may extend to an outer circumference of the fixed cutting element holder.

In certain embodiments, the elongated housing may comprise an opening at the distal end and the rotating cauterizing element may be configured to rotate from a first position to a second position, where the first position does not cover the opening at the distal end and where the second position covers the opening at the distal end. Particular embodiments may also comprise a vacuum source configured to create a vacuum within the housing.

Specific embodiments may include a method of removing tissue, where the method comprises: placing a tissue removal device proximal to a section of tissue; inserting the section of tissue into the distal end of the elongated housing; and moving the rotating cauterizing element from a first position to a second position to remove the section of tissue from a target site. In particular embodiments, the tissue removal device may comprise: an elongated housing comprising a proximal end and a distal end; an electric motor; a fixed cauterizing element proximal to the distal end of the housing; and a rotating cauterizing element proximal to the distal end of the housing, wherein the electric motor is coupled to the rotating cauterizing element.

Certain embodiments may also comprise supplying electric power to the fixed cauterizing element. Specific embodiments may also comprise transmitting electric power from the fixed cauterizing element to the rotating cauterizing element. In certain embodiments, the electric power may be sufficient to cauterize tissue at the target site. In particular embodiments, the electric power may be sufficient to coagulate blood vessels at the target site. In specific embodiments, inserting the section of tissue into the distal end of the elongated housing comprises applying a vacuum device to the housing to draw the section of tissue into the housing. In particular embodiments, the tissue may comprise lymphatic tissue. In certain embodiments, the tissue may comprise fat tissue.

Embodiments of the invention are directed to devices and methods that improve the surgical efficiency and shorten the operation time for tissue removal. To serve this purpose, a tissue removal device is designed using both mechanical dissection and suction to grasp, resect and collect all or part of a target tissue. In exemplary embodiments, heating elements can be included for tissue removal and/or cauterization to stop bleeding, as explained in further detail below. The device described herein can be used in a wide range of tissue removal surgeries including both open and laparoscopic surgery. Also, the device is capable of removing other kinds of tissues such as fat and skin tissues, in addition to lymphatic tissues. Use of the device described herein renders the tissue removal methods significantly faster than the conventional methods of surgery. The device is configured for use in both open and laproscopic surgery, and the shortened surgery times will subsequently reduce the time for patients under anesthesia and make it possible for the surgeon to work with ease and efficiency using minimally invasive techniques.

In the following, the term “coupled” is defined as connected, although not necessarily directly, and not necessarily mechanically.

In certain aspects, the surgery is carried out in the affected tissues in which contact between the other body parts and the target need to be minimized The current device is designed in a way to minimize bleeding and inadvertent damage with adjacent viable tissue.

Certain embodiments are directed to a device for resecting a target tissue comprising: (a) a mechanical cutting head having an exterior face and an interior face, (i) the cutting head comprising at least two fixed blades, the fixed blades forming flow channels in the cutting head; and (ii) one or more movable blades positioned interior to the fixed blades of the cutting head; and (b) a mounting head coupled to and fluidically connected to the cutting head, the mounting head being configured to provide a suction force to draw the target tissue inside the cutting head where the target tissue is cut by the moveable blades and fixed blades during use.

The device can further comprising heating elements positioned on or in the fixed blades. In certain aspects, the heating elements are capable of cauterizing tissue.

In certain aspects, the cutting head is 1, 2, 3, 4, 5, 6, 7, 8, 9, or 10 mm to 8, 9, 10, 11, 12, 13, 14, or 15 mm, including all values and ranges there between, in diameter or at its maximum width. In a further aspect, the cutting head can be 1, 2, 3, 4, 5, 6, 7, 8, 9, or 10 cm to 8, 9, 10, 11, 12, 13, 14, or 15 cm, including all values and ranges there between, in diameter or at its maximum width. In a particular aspect, the cutting head is 3 mm to 10 mm in diameter or at its maximum width. In certain aspects, the cutting head is 10 mm in diameter or at its maximum width.

In certain aspects, the device comprises 1, 2, 3, 4, 5, 6, 7, 8, 9, 10 or more fixed blades. In a further aspect, the device comprises 4 fixed blades. In a further aspect, the device has at least the same number of moveable as it does fixed blades. In a further aspect, the device has 2, 3, 4, 5, 6, 7, 8, 9, 10, 11, or more moveable blades. In certain aspects, the device has at least 4 moveable blades.

In certain aspects, the fixed blades have an angle of between 55 and 60 degrees relative to the long axis of the device. In a further aspect, the fixed blades have an angle of between 57 and 58 degrees relative to the long axis of the device. In still a further aspect, the fixed blades have an angle of about 57.5 or 57.47 degrees relative to the long axis of the device.

Certain embodiments are directed to an apparatus for resecting a target tissue comprising a device of claim 1 operatively coupled to a vacuum source and a collection container.

Further embodiments are directed to a fixed blade assembly having an optimized flow velocity through the assembly comprising four or more fixed blades having an angle of 55 to 60 degrees relative to the short axis of the assembly. In certain aspects, the blade angle is between 57 and 58 degrees relative to the short axis of the assembly. In further aspects, the blade angle is or is about 57.47 degrees relative to the short axis of the assembly. In still a further aspect, the assembly has 1, 2, 3, 4, 5, 6, 7, 8, 9, 10 or more fixed blades. In certain aspects, the assembly has a maximum width of 3, 4, 5, 6, 7, 8, 9, or 10 mm. In a further aspect, the assembly has a maximum width of 1, 2, 3, 4, 5, 6, 7, 8, 9, or 10 cm

Certain embodiments are directed to methods of resecting a target tissue comprising inserting a device as described herein in to a patient in need of target tissue removal and contacting the target tissue with the device, wherein the target tissue is grasped by the device, drawn inside the device, cut from the patient, and collected. In certain aspects, the target tissue is lymphatic, fat, or skin tissue. In certain aspects, the device is inserted by open or laparoscopic surgery.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention

The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will be apparent to those skilled in the art from this detailed description.

### BRIEF DESCRIPTION OF THE DRAWINGS

The following drawings form part of the present specification and are included to further demonstrate certain aspects of the present disclosure. The invention may be better understood by reference to one of these drawings in combination with the detailed description of specific embodiments presented herein.

FIG. 1 shows a perspective view according to an exemplary embodiment.

FIG. 2 shows an exploded view of the embodiment of FIG. 1.

FIG. 3 shows a section view of the embodiment of FIG. 1.

FIG. 4 shows a partial perspective view of the embodiment of FIG. 1.

FIG. 5 shows a perspective view of a first component of the embodiment of FIG. 1.

FIG. 6 shows a perspective view of a second component of the embodiment of FIG. 1.

FIG. 7 shows a perspective view of a third component of the embodiment of FIG. 1.

FIG. 8 shows a perspective view of a fourth component of the embodiment of FIG. 1.

FIG. 9 shows a perspective view of the components of FIGS. 5-8 in multiple positions.

FIG. 10 shows a perspective view of the embodiment of FIG. 1.

FIG. 11 illustrates an external three-dimensional view of one embodiment of a tissue removal device.

FIG. 12 illustrates an external side view of one embodiment of a tissue removal device.

FIG. 13 illustrates an external top view of one embodiment of a tissue removal device.

FIG. 14 illustrates an inside view of one embodiment of a tissue removal device showing moving blades, inlets, and outlets

FIG. 15 illustrates a cross-sectional side view of a tissue removal device.

FIG. 16 illustrates an example of an enclosure of one embodiment of a tissue removal device.

FIG. 17 illustrates one example of a moving blade assembly in blade mount of a tissue removal device.

FIG. 18 illustrates one example of an actuator mount of a tissue removal device.

FIG. 19 illustrates an example of an actuator of a tissue removal device.

FIG. 20 illustrates an example of a moving blade mount of a tissue removal device.

FIG. 21 illustrates an example of a cam of a tissue removal device.

FIG. 22 illustrates an example of a fixed blade assembly of a tissue removal device.

FIG. 23 illustrates an example of a head cover of a tissue removal device.

FIG. 24 illustrates an example of a head mount of a tissue removal device.

FIG. 25 illustrates an example of a pneumatic adapter of a tissue removal device.

FIG. 26 illustrates one example of shape optimization of the fixed blades of a tissue removal device.

FIG. 27 illustrates one example of the fixed blade configuration.

### DETAILED DESCRIPTION OF ILLUSTRATIVE EMBODIMENTS

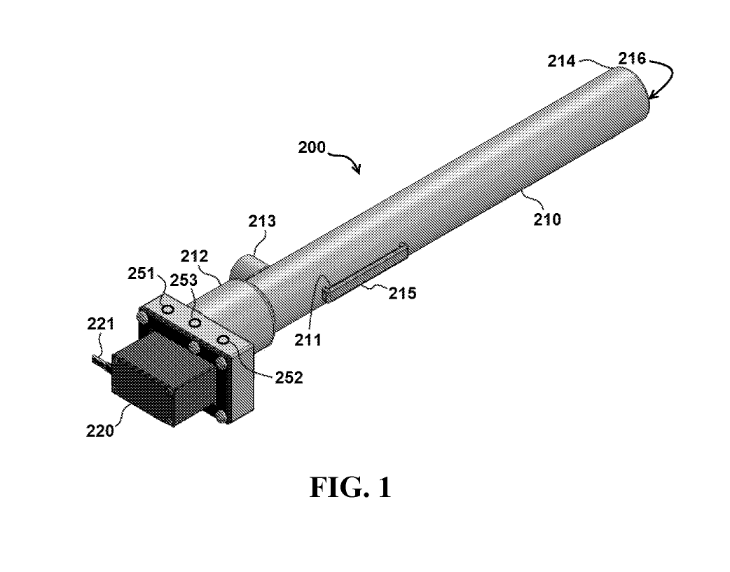

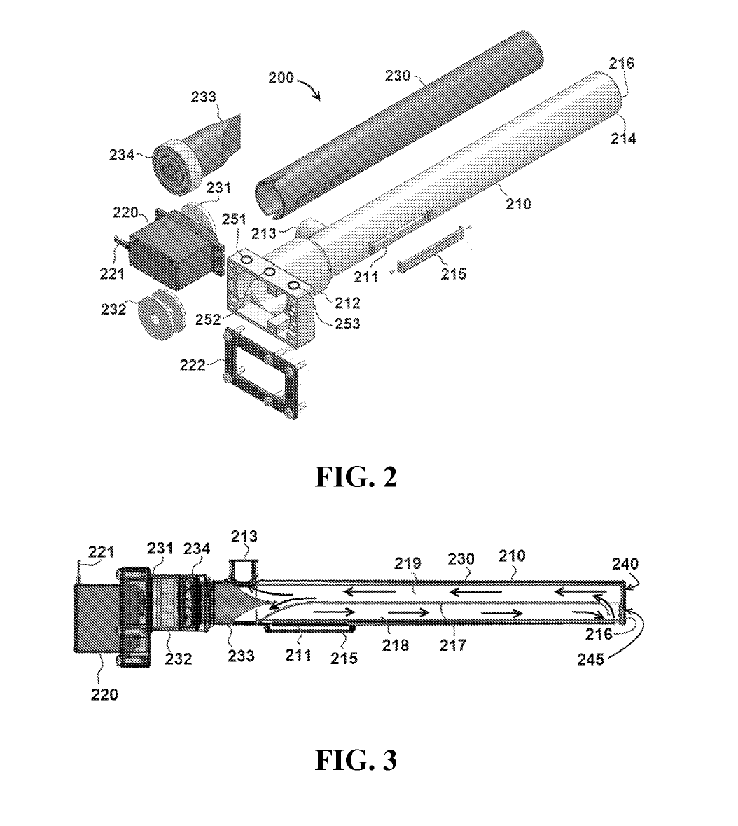

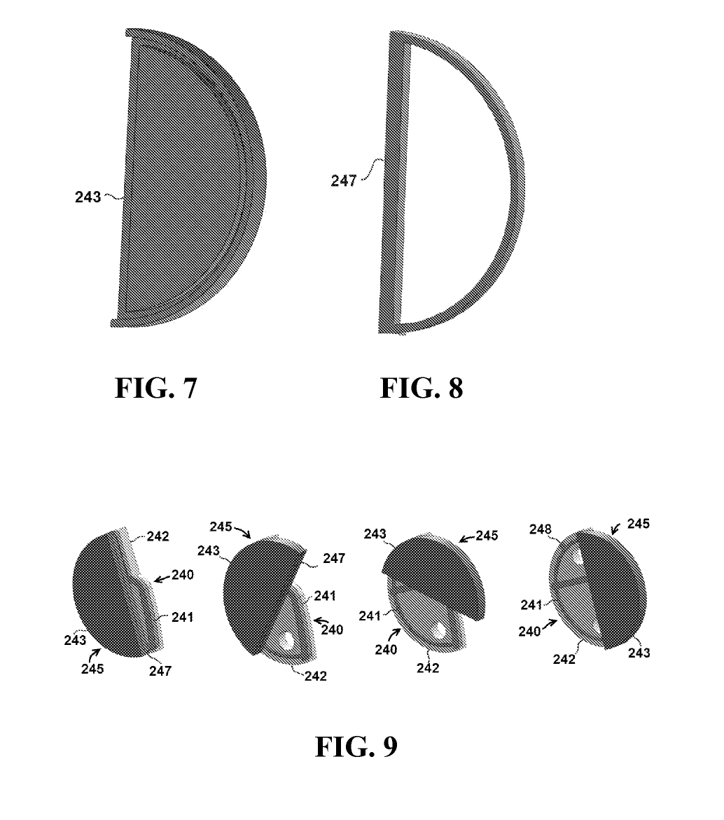

Referring initially to FIGS. 1-10, an exemplary embodiment of a tissue removal device 200 comprises a housing 210 with a proximal end 212, a distal end 214, and an air inlet 211 with a cover 215. As shown more clearly in the exploded view of FIG. 2, tissue removal device 200 further comprises a motor 220 coupled to a drive extension 230 via a drive disc 231, a coupling mechanism 232, and a drive member 233 that is supported by a bearing 234. In the illustrated embodiment, motor 220 is also coupled to housing 210 via a mounting plate 222. Electrical power and control signals (e.g. via a microcontroller) can be supplied via wiring 221 to motor 220 and other components of tissue removal device 200 requiring electrical power, e.g. cauterizing elements or vacuum devices described in further detail below. It is understood that the specific configuration shown in the figures represents merely one exemplary embodiment of a tissue removal device according to the present disclosure, and that other exemplary embodiments may comprises a different configuration or combination of components. In addition, for purposes of clarity, not all components are labeled with reference numbers in each view.

As will be explained in further detail below, housing 210 is generally cylindrical in shape and further comprises an inlet 216 near distal end 214. Housing 210 also comprises an outlet 213 located near proximal end 212. Inlet 216 can allow air and the tissue to be removed to enter housing 210, while outlet 213 can allow air and the tissue that has been removed from a target site to exit housing 210.

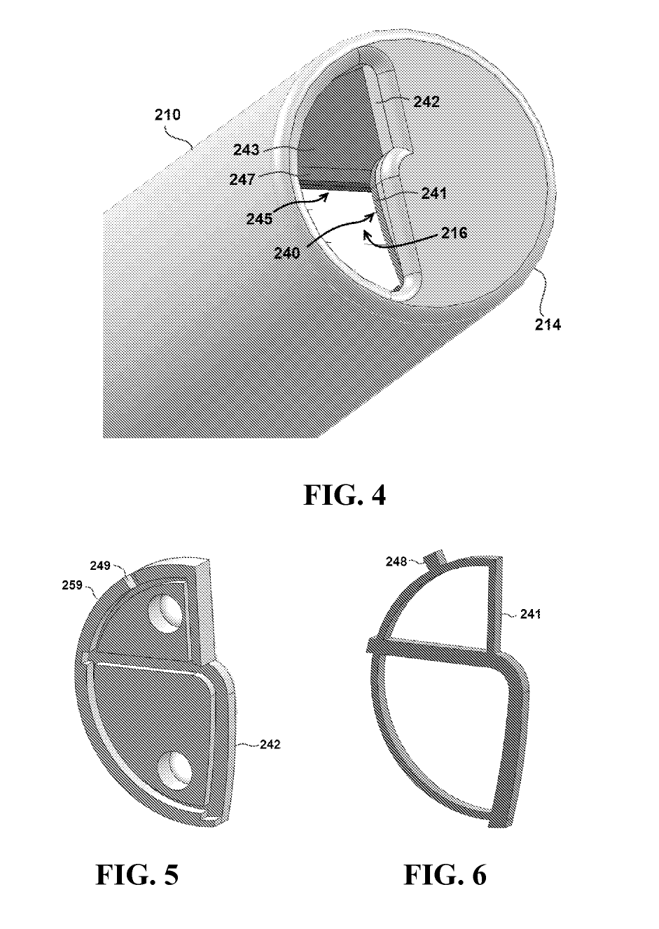

Referring now to FIGS. 3-4, a section view and an end perspective view illustrate the flow of air and/or tissue that can occur during operation. As shown in the section view of FIG. 3, housing 210 comprises a partition 217 that divides the interior volume of housing 210 into two compartments 218 and 219. During operation, a low pressure or vacuum can be created in compartment 219 so that air flows into air inlet 211 and through compartment 218 to compartment 219. In specific embodiments, a vacuum device (e.g. a vacuum pump) can be coupled to outlet 213 of housing 210. This low pressure or vacuum within housing 210 can assist in drawing or pulling the tissue to be removed into housing 210 at inlet 216. In particular embodiments, a sensor may be placed near distal end 214 to determine the position of tissue that is drawn into inlet 216. In certain embodiments, tissue removal device 200 may comprise a positive pressure device (e.g. an air pump) to push tissue towards distal end 214 in the event that it is drawn further than desired into housing 210. When the tissue is placed in the desired location near distal end 214, it can then be excised and/or cauterized as explained in more detail below. For example, the tissue to be removed can then be excised by a shearing action created between a fixed cutting element 241 and a rotating cutting element 247, as explained further below.

Referring specifically now to FIGS. 4-9, in this embodiment fixed cauterizing element 240 and a rotating cauterizing element 245 are located proximal to distal end 214 of housing 210. Fixed cauterizing element 240 comprises a fixed cutting element 241 that is electrically conductive in a fixed element holder 242 that is not electrically conductive. In addition, rotating cauterizing element 245 comprises a rotating cutting element 247 that is electrically conductive in a rotating element holder 243 that is not electrically conductive. In this embodiment, fixed cutting element 241 is in contact with rotating cutting element 247 throughout the rotation of rotating cutting element 247.

FIG. 9 illustrates rotating cauterizing element 245 in four different positions during rotation. In the first position on the left, opening 216 at distal end 214 (shown in FIG. 4) is fully open, while in the second and third positions opening 216 is partially closed. In the fourth position shown on the far right, rotating cauterizing element 245 has rotated 180 degrees from the initial position and opening 216 would be fully closed. As shown in FIG. 9, rotating cutting element 247 is immediately adjacent and in contact with fixed cutting element 241 so that the configuration provides a shearing action between the cutting elements during rotation.

Referring back now to FIG. 4, rotating cauterizing element 245 and rotating cutting element 247 are shown in a position that has rotated approximately 90 degrees from the initial position in which opening 216 is fully open. As rotating cauterizing element 245 continues rotation, opening 216 will be reduced in size until it is fully closed. Any tissue that is inserted into opening 216 will therefore be sheared or cut by rotating cutting element 247 and fixed cutting element 241.

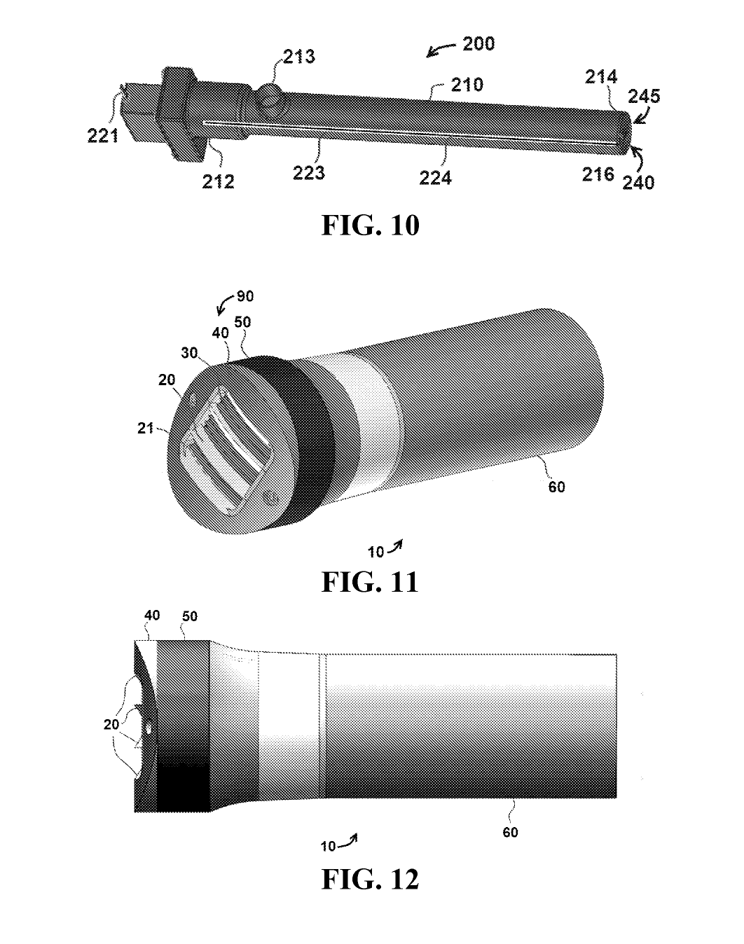

As shown in FIG. 10, in this embodiment an electric power source (e.g. an alternating current source, battery pack, or other source) can be electrically coupled to the fixed cutting element 241 via wiring 221 and a wire 223 that extends in a channel 224 along the primary length of housing 210. Wire 223 can be coupled to an extension 248 (shown in FIG. 6) of fixed cutting element 241. When assembled, extension 248 extends through a slot 249 (shown in FIG. 5) to an outer circumference 259 of fixed element holder 242.

In certain embodiments, tissue removal device 200 can be configured to provide radio frequency electric power to the fixed cutting element 241. In the embodiment shown, tissue removal device 200 can comprise a first control switch 251 configured to supply electrical power to the fixed cutting element 241 at a first power level sufficient to cauterize lymphatic tissue. Tissue removal device 200 can further comprise a second control switch 252 configured to supply electrical power to fixed cutting element 241 at a second power level sufficient to coagulate blood vessels. As previously mentioned fixed cutting element 241 and rotating cutting element 247 are in contact with each other and are electrically conductive. Accordingly, the electrical power supplied to fixed cutting element 241 is also conducted to rotating cutting element 247. This configuration allows both fixed cutting element 241 and rotating cutting element 247 to cauterize tissue and/or coagulate blood vessels.

In the embodiment shown, a third control switch 253 can be used to control the level of vacuum applied to compartment 219 in housing 210. It is understood that the location and configuration of control switches 251, 252 and 253 are shown for example only, and that other exemplary embodiments may comprise a different location and configuration. For example, in certain embodiments, tissue removal device 200 may comprise an ergonomic handle that incorporates the control switches.

The exemplary embodiment shown and described in FIG. 1-10 can allow a user to remove tissue from a target site with a precise cutting action that preserves the removed tissue intact and allows it to be analyzed at a later time. It can also allow a user to accurately control the level of electrical power applied to the cutting elements, which can be used for both cauterization and coagulation. This can provide the user with the flexibility to perform either cauterization or coagulation with a single instrument. The features shown and described in this exemplary embodiment allow a user to perform tissue removal procedures efficiently and accurately.

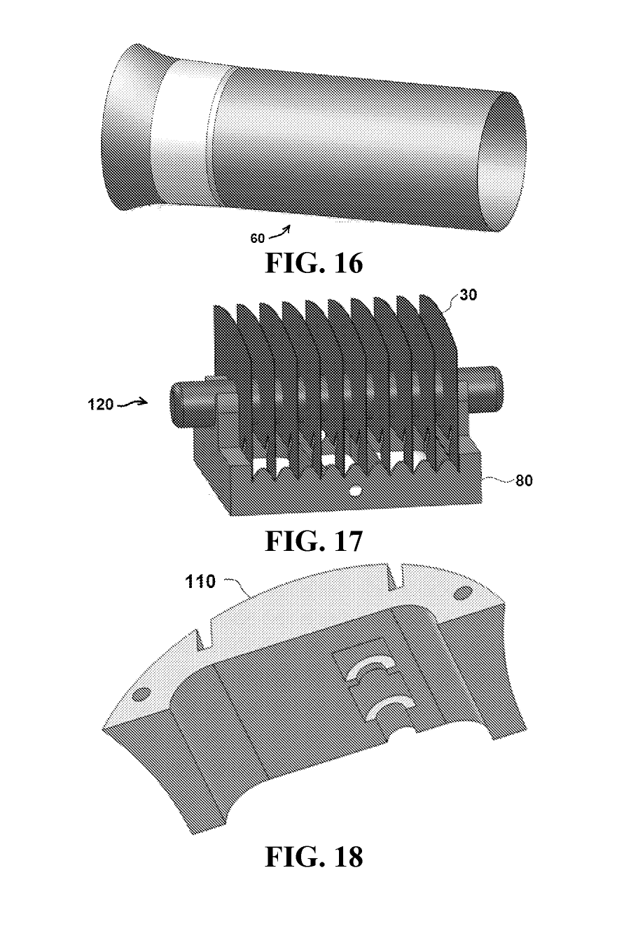

Referring now to FIGS. 11-27, another exemplary embodiment of a tissue removal device is illustrated. Certain embodiments of the device can comprise multiple systems, including cutting blades, vacuum apparatus, water pump, and alternatively heating elements.

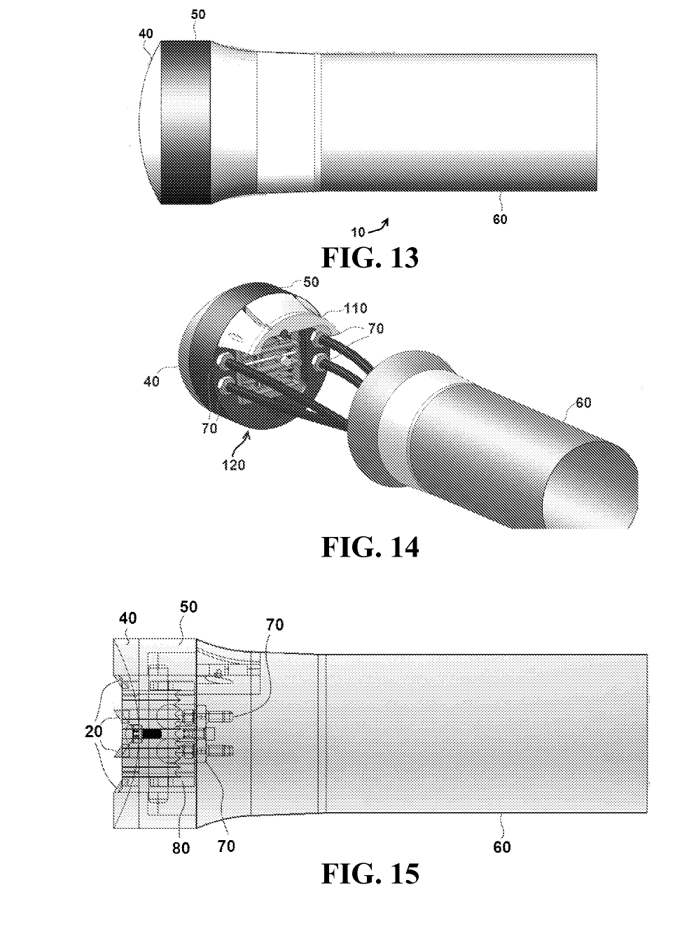

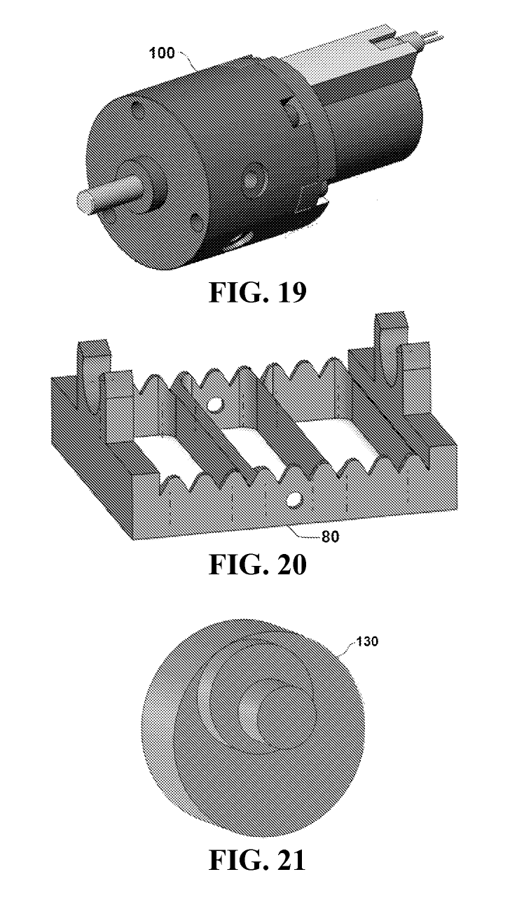

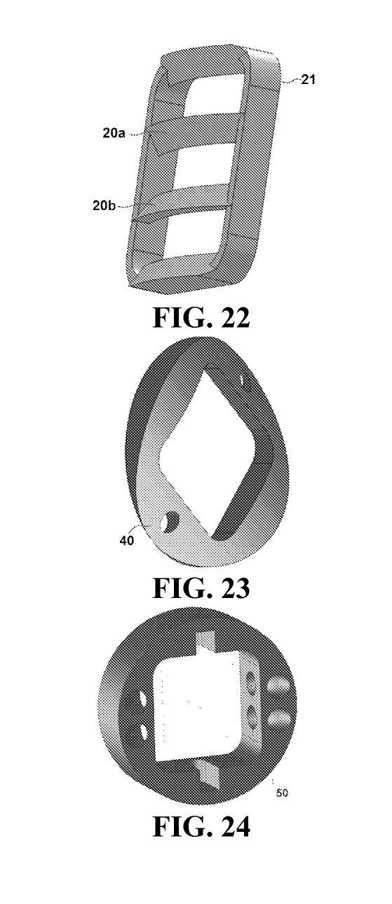

FIG. 11 illustrates one embodiment of an assembled tissue removal device 10. The cutting head 90 that contains the cutting blades (fix blades 20 and movable blades 30) is positioned at the tip or distal end of the long axis of the device 10. In certain aspects, the cutting head has a diameter of 3 mm to 10 mm. In certain aspects the diameter of the cutting head is about 10 mm. In a further aspect the cutting head may have a diameter of 10 mm to 10 cm. The fixed blades assembly 21, which includes fix blades 20, is attached with the head cover 40 and then the combined structure is placed at the distal end of the enclosure 60 relative to the long axis of the device, the opposite end of the device being the proximal end. The device can contain 2, 3, 4, 5, 6 or more fixed blades that are configured to optimize velocity of flow through flow channels formed in the cutting head, optimizing the grasping or suction force applied to a target tissue during use of the device. In certain embodiments, the number of fixed blades can be chosen to be between four and the total number of moving blades. Fixed blades 20 face the opposite direction of the moveable blades 30. Fixed blades 20 all have the same angle. In certain aspects the angle of the fixed blade from the horizontal is 50, 52, 53, 54, 55, or 56 to 57, 58, 59, or 60 degrees, including all values and ranges there between. In a further aspect, the fixed blade angle is in the range of 57 to 58 degrees. In particular aspects the fixed blade angle is 57.46 degrees from the horizontal (FIG. 27). In certain aspects heating elements are incorporated or attached to the fixed blades. In a further aspect, the heating element(s) configured as cauterizing agent(s). The moving blades 30 are mounted in the blade mount 80 (see FIG. 17 and FIG. 20) that is attached to the head mount 50 (FIG. 24). The moving blade assembly 120 (FIG. 17, comprising moving blades 30 mounted on moving blade mount 80) is positioned proximal to the fixed blades assembly 21 (FIG. 22) and configured to cut target tissue that enters the cutting head through one or more flow channels (i.e. the space formed by fixed blades in the fixed blade assembly) in the cutting head. The actuator 100 (FIG. 19) attached to the actuator mount 110 (FIG. 18), is positioned proximal to the moving blade assembly 120 (FIG. 17) such that it can move movable blades 30 using cam 130 (FIG. 21). In certain aspects the moveable blade(s) reciprocate, rotate, or scissor. In certain aspects, the blades are manufactured from medical grade metals or metal alloys approved by FDA. FIG. 12 illustrates an external side view of tissue removal device 10. FIG. 13 illustrates an external top view of tissue removal device 10. A tubular member can be connected to the mounting head. The tubular member can be configured to provide vacuum force to the mounting and/or a path for tissue scraps and/or body fluids.

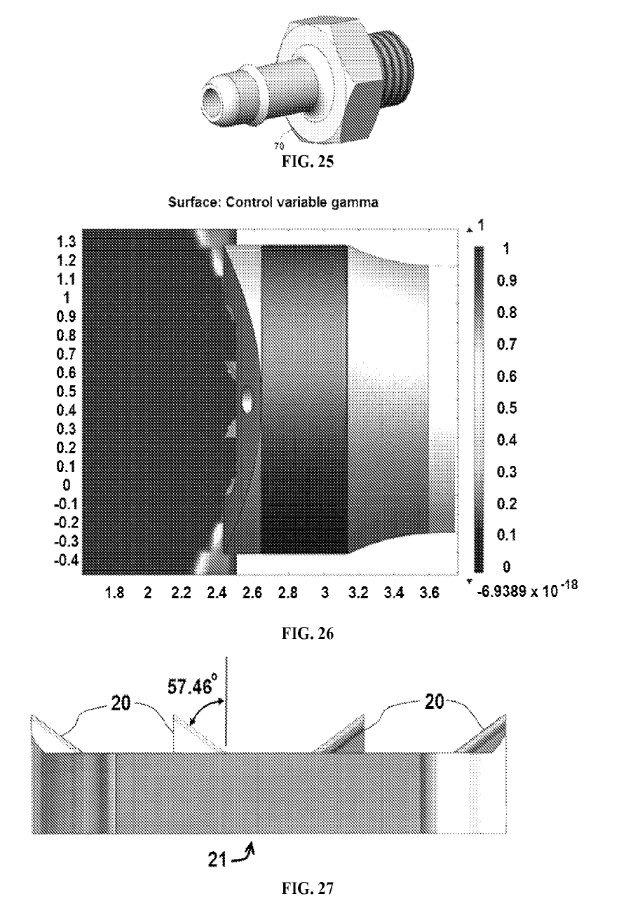

FIG. 14 provides a partially exploded view of tissue removal device 10. The flow channel(s) formed in the fixed blade assembly 21 are fluidically connected to the head mount 50 (FIG. 14) and a vacuum source and/or a fluid source (e.g., water source) through pneumatic adapters 70 (FIG. 25). A vacuum pump and water pump can be connected through separate pneumatic adapters so that they can act separately and create suction pressure. Alternatively the vacuum source can be a single multiphase pump. In certain aspects, one or more flow regulating valves and/or stop valves are positioned in the tubing or connection from a fluid and/or vacuum source.

When suction is applied the fluid source is turned off and the stop valve at the fluid adapter is kept close. As a result, suction force is generated at the distal tip of the device that functions to draw tissue through the flow channels and inside the device. The shape of the fixed blades is optimized for maximizing the flow velocity near the distal tip of the device (FIG. 26). Angles of fixed blades, thickness of each blade and the distance between blades are chosen, but not limited to these three, to be shape and topological parameters that are optimized. The resulting optimal design is significantly superior in comparison to the straightforward design using vertical angles with equal spacing. Also, the number of fixed blades greater than four but less than or equal than the number of moving blades, which is designed to be more than the number of fixed blades. As a target tissue enters the device it is cut by the blades. In certain aspects, a reciprocating moving blade motion is created by an actuator/cam assembly. In a further aspect, heating elements attached to or incorporated in the fixed blades helps to mitigate blood spatter. The vacuum force also helps to prevent blood from escaping through the distal tip of the device and directs flow to a collection container. Tissue scraps are sucked by the vacuum and collected. In certain aspects a collection container is incorporate into the device and is fluidically connected to mount head producing tissue scraps and fluid entering the device. In a further aspect, a collection container can be positioned externally relative to a patient and fluidically connected via tubes or the like.

In certain aspects the device can be operated in cleaning mode. The cleaning mode can be used with the device inside or outside the body of a patient. The cleaning mode is initiated if any scrap accumulates and jams the passage through the moving blades. In certain aspects a flow regulating valve and/or stop valve can be opened for cleaning purposes allowing a fluid to flow. The fluid is then sucked in to the head mount. In certain aspects the cleaning fluid (e.g., air) can be pumped in using a pressure source (e.g., air blower). The fluid can dislodge or move any accumulated tissue scraps from the moving blades and make the device re-usable. In certain aspects the device is used in a continuous mode where a number of target tissue sites can be removed without removing and re-inserting the device. In certain aspects the cleaning fluid can also offer lubrication. In certain aspects, the cleaning fluid can also be used as a diluting agent.

All of the apparatus, systems and/or methods disclosed and claimed herein can be made and executed without undue experimentation in light of the present disclosure. While the devices, systems and methods of this invention have been described in terms of particular embodiments, it will be apparent to those of skill in the art that variations may be applied to the devices, systems and/or methods in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

The contents of the following references are incorporated by reference herein:
U.S. Patent Publication 2003/0125768
U.S. Patent Publication 2012/0022323
U.S. Pat. No. 3,954,102
U.S. Pat. No. 4,009,711
U.S. Pat. No. 4,201,202
U.S. Pat. No. 4,318,396
U.S. Pat. No. 4,342,308
U.S. Pat. No. 4,407,278
U.S. Pat. No. 5,062,416
U.S. Pat. No. 6,475,137
U.S. Pat. No. 7,011,624
U.S. Pat. No. 7,367,938
U.S. Pat. No. 7,338,437
U.S. Pat. No. 7,407,481
U.S. Pat. No. 7,442,165
U.S. Pat. No. 8,126,558
U.S. Pat. No. 8,290,594
U.S. Pat. No. 8,287,444

1-41. (canceled)

42. A tissue removal device comprising:
an elongated housing comprising a proximal end and a distal end;
an electric motor;
a fixed cauterizing element proximal to the distal end of the housing; and
a rotating cauterizing element proximal to the distal end of the housing, wherein the electric motor is coupled to the rotating cauterizing element.

43. The tissue removal device of claim 42 wherein the electric motor is coupled to the rotating cauterizing element via a drive extension within the elongated housing.

44. The tissue removal device of claim 43 further comprising a drive member coupling the electric motor and the drive extension.

45. The tissue removal device of claim 42 wherein:
the fixed cauterizing element comprises a fixed cutting element that is electrically conductive;
the rotating cauterizing element comprises a rotating cutting element that is electrically conductive;
and the fixed cutting element is in contact with the rotating cutting element.

46. The tissue removal device of claim 45 wherein the tissue removal device is configured so that an electric power source can be electrically coupled to the fixed cauterizing element; and the tissue removal device further comprises a first control switch configured to supply electrical power to the fixed cutting element at a first power level sufficient to cauterize tissue.

47. The tissue removal device of claim 46 comprising:
a first control switch configured to supply electrical power to the fixed cutting element at a first power level sufficient to cauterize tissue;
and a second control switch configured to supply electrical power to the fixed cutting element at a second power level sufficient to coagulate blood vessels.

48. The tissue removal device of claim 46 wherein:
the rotating cauterizing element comprises a rotating cutting element holder that is not electrically conductive and wherein the fixed cauterizing element comprises a fixed cutting element holder that is not electrically conductive.
and wherein the fixed cutting element extends to an outer circumference of the fixed cutting element holder.

49. The tissue removal device of claim 45 wherein:
the elongated housing comprises an opening at the distal end;
the rotating cauterizing element is configured to rotate from a first position to a second position;
the first position does not cover the opening at the distal end;
and the second position covers the opening at the distal end.

50. The tissue removal device of claim 42 further comprising a vacuum source configured to create a vacuum within the housing.

51. A method of removing tissue, the method comprising:
placing a tissue removal device proximal to a section of tissue, wherein the tissue removal device comprises:
an elongated housing comprising a proximal end and a distal end;
an electric motor;
a fixed cauterizing element proximal to the distal end of the housing;
and a rotating cauterizing element proximal to the distal end of the housing, wherein the electric motor is coupled to the rotating cauterizing element; inserting the section of tissue into the distal end of the elongated housing; and
moving the rotating cauterizing element from a first position to a second position to remove the section of tissue from a target site.

52. The method of claim 51 further comprising supplying electric power to the fixed cauterizing element.

53. The method of claim 52 further comprising transmitting electric power from the fixed cauterizing element to the rotating cauterizing element.

54. The method of claim 51 wherein inserting the section of tissue into the distal end of the elongated housing comprises applying a vacuum device to the housing to draw the section of tissue into the housing.

55. A device for resecting a target tissue comprising:
(a) a mechanical cutting head having an exterior face and an interior face,
(i) the cutting head comprising at least two fixed blades, the fixed blades forming flow channels in the cutting head; and
(ii) one or more movable blades positioned interior to the fixed blades of the cutting head; and
(b) a mounting head coupled to and fluidically connected to the cutting head, the mounting head being configured to provide a suction force to draw the target tissue inside the cutting head where the target tissue is cut by the moveable blades and fixed blades during use.

56. The device of claim 55, further comprising heating elements position on or in the fixed blades.

57. The device of claim 55, wherein the cutting head is 3 mm to 10 mm in diameter.

58. The device of claim 55, wherein the fixed blades have an angle of between 55 and 60 degrees relative to the long axis of the device.

59. The device of claim 55, wherein the fixed blades have an angle of between 57 and 58 degrees relative to the long axis of the device.

60. The device of claim 55, wherein the fixed blades have an angle of about 57.5 degrees relative to the long axis of the device.

61. An apparatus for resecting a target tissue comprising a device of claim 55 operatively coupled to one or more of a vacuum source, a fluid source, a pressure source, a heat source, an electrical source, and/or a collection container.
